(19) United States
(12) Patent Application Publication
(10) Pub. No.: US 2019/0328591 A1
(43) Pub. Date: Oct. 31, 2019

(54) APPARATUS AND METHOD FOR PROCESSING AND PACKAGING DISPOSABLE PRODUCTS

(71) Applicant: Curt G. Joa, Inc., Sheboygan Falls, WI (US)

(72) Inventors: Collin M. Heinz, Plymouth, WI (US); Robert E. Andrews, Sheboygan, WI (US); Cory D. Veldman, Plymouth, WI (US); Kevin D. Blank, Port Washington, WI (US)

(21) Appl. No.: 16/395,551

(22) Filed: Apr. 26, 2019

Related U.S. Application Data
(60) Provisional application No. 62/663,442, filed on Apr. 27, 2018.

(51) Int. Cl. A61F 13/551; B65B 35/58; B65B 35/50

(52) U.S. Cl. CPC A61F 13/55105; B65B 35/50; B65B 35/58

(57) ABSTRACT

A method for reorienting discrete hygiene products for packaging includes providing discrete products traveling in a machine direction and all in a first orientation to a product turner and placer comprising a plurality of pucks rotatable about a central axis, with each puck also rotatable about a spin axis. The method also includes receiving a discrete product on each puck, transferring the discrete products via the pucks from a pick-up location to a drop-off location, and depositing the discrete products from the pucks onto a conveyor at the drop-off location, the discrete products deposited so as to be spaced apart on the conveyor. In transferring and depositing the discrete products, the rotation of each of the pucks is selectively controlled about its spin axis to reorient each of the discrete products for depositing onto the conveyor, with each of a direction and an amount of the rotation being selectively controlled.

### CROSS-REFERENCE TO RELATED APPLICATION

The present invention is a non-provisional of and claims priority to U.S. Provisional Patent Application Ser. No. 62/663,442, filed Apr. 27, 2018, the disclosure of which is incorporated herein by reference in its entirety.

### BACKGROUND OF THE INVENTION

The present invention relates to disposable hygiene products and more specifically, to methods and apparatuses for processing disposable hygiene products such as baby diapers, adult diapers, disposable undergarments, incontinence devices, sanitary napkins and the like. Still more specifically, the invention relates to an apparatus and method for mechanically manipulating traveling discrete hygiene products to re-position them for packaging, to thereby reduce overall packaging volume and also reduce building manufacturing space.

Generally, diapers comprise an absorbent insert or patch and a chassis, which, when the diaper is worn, supports the insert proximate a wearer's body. A representative diaper chassis comprises a nonwoven web material and support structure. The diaper support structure is generally elastic and may include leg elastic, waistband elastic and belly band elastic. The support structure is usually sandwiched between layers of the nonwoven web material, which is fed from material rolls, through splicers and accumulators. The chassis may also be provided with several patches, besides the absorbent insert. Representative patches include adhesive tape tabs and resealable closures. The absorbent insert may be composed of cellulose pulp core that includes a super-absorbent powder that functions to absorb exudates to keep the wearer dry, with a tissue web being wrapped around the pulp core.

The diapers may be assembled by adding components to and/or otherwise modifying an advancing, continuous web of material. In some processes, advancing webs of material are combined with other advancing webs of material. In other processes, individual components created from advancing webs of material are combined with advancing webs of material, which in turn, are then combined with other advancing webs of material. Webs of material and component parts used to manufacture diapers may include: backsheets, topsheets, leg cuffs, waist bands, absorbent core components, front and/or back ears, front and/or back belts, fastening components, and various types of elastic webs and components such as leg elastics, barrier leg cuff elastics, stretch side panels, and waist elastics. Upon application and assembling of all desired components/materials in a diaper, a discrete diaper may be cut/formed and then folded to bring front and back waist regions into a face-to-face relationship, with the diaper then subsequently being advanced to a packaging operation.

In the production of diapers (and other disposable hygiene products), there is an advantage to alternating the orientation of every other diaper for packaging to save bag space by creating uniform density of pack volume. A single diaper (or other disposable hygiene product) is typically thicker on the fold side due to the additional bulk of the core. Since the open end of the diaper lacks a core to the extreme edge of the product, that end is typically not as thick as the fold side. Therefore, alternating the position of the diapers such that thick and thin parts of the product nest together, provides a more compact and efficient packaging.

However, despite these benefits of alternating the positioning of diapers in a packaging operation, existing packaging systems package all diapers in a common or same orientation. An example of such a system 2 is illustrated in FIG. 1, where an incoming web of continuous formed and sideseam bonded diapers 4 is shown entering a vertically oriented cutting unit 6. The cutting unit 6 operates to sever the bonded diapers to separate them into discrete diapers, with the diapers being transferred to a product turner and placer 8. The product turner and placer 8 pivots the diapers from a vertical orientation to a horizontal orientation, and places all diapers onto a horizontal conveyor 10 such that they are positioned in a common/same direction and orientation. The diapers are then translated down the horizontal conveyor 10, are subsequently turned from the horizontal orientation back to a vertical orientation, and are then provided to a packaging system (not shown) for packaging.

The system 2 of FIG. 1 thus operates to output diapers to a packaging system with all diapers being supplied in a common orientation/direction, such that the thicker core portion of the diapers are all stacked on top of one another when packaged. This packaging can lead to an increased package height and asymmetry in the final package of diapers. Another drawback to the system of FIG. 1 is that the footprint of the system 2 is unnecessarily large, as space is required on the conveyor 10 to receive the diapers in a horizontal orientation and then subsequently perform a turning operation to re-orient them back to a vertical orientation prior to the packaging thereof, which is an inefficient operation that adds processing steps and adds to the length of the conveyor.

Accordingly, it is desirable to provide an improved apparatus and method for processing and positioning discrete hygiene products for packaging in an efficient manner. Such a system and method would shorten the packaging line so as to reduce the packaging system footprint. Such a system and method would also be selectively configurable to alternate the orientation of products in different manners, so as to accommodate different diaper sizes and/or different product types. For example, for one product it may be desirable to rotate every other product with +90 and −90 orientations, while in another product it may be desirable to rotate every other product with 0 and 180 orientations, while in still another product it may be desirable to rotate every product with a +90 orientation. Currently, there is no existing system or method that would allow for selection and customization of controlling rotation of a product for packaging.

### BRIEF DESCRIPTION OF THE INVENTION

In accordance with one aspect of the invention, a method for reorienting discrete hygiene products for packaging is provided. The method includes providing discrete hygiene products traveling in a machine direction and all in a first orientation to a product turner and placer, the product turner and placer comprising a plurality of pucks attached to a puck wheel so as to be rotatable about a central axis thereof, with each puck also rotatable about a spin axis thereof. The method also includes receiving a discrete hygiene product on each respective puck, transferring the discrete hygiene products via the plurality of pucks from a pick-up location to a drop-off location, and depositing the discrete hygiene products from the plurality of pucks onto a conveyor at the drop-off location, the discrete hygiene products deposited so as to be spaced apart on the conveyor. In transferring and depositing the discrete hygiene products, the method further comprises selectively controlling the rotation of each of the plurality of pucks about its spin axis to reorient each of the discrete products for depositing onto the conveyor, with each of a direction and an amount of the rotation being selectively controlled.

In accordance with another aspect of the invention, a discrete hygiene product processing system includes an input device configured to translate discrete hygiene products in a machine direction and a product turner and placer positioned to receive the discrete hygiene products from the input device. The product turner and placer further includes a puck wheel rotatable about a central axis and a plurality of pucks attached to the puck wheel so as to be rotatable therewith, with each of the plurality of pucks configured to discretely rotate about a spin axis and configured to receive a discrete hygiene product from the input device. The discrete hygiene product processing system also includes a conveyor positioned adjacent the product turner and placer to receive discrete hygiene products as they are released from the plurality of pucks. Each of the plurality of pucks is selectively controllable to rotate about its spin axis to reorient each of the discrete products for depositing onto the conveyor.

These and other advantages and features will be more readily understood from the following detailed description of preferred embodiments of the invention that is provided in connection with the accompanying drawings.

### BRIEF DESCRIPTION OF THE DRAWINGS

The drawings illustrate embodiments presently contemplated for carrying out the invention.

In the drawings:

FIG. 1 is a perspective view of a processing system for providing discrete hygiene products for packaging, as known in the prior art.

FIG. 2 is a perspective view of a hygiene product for use with the present apparatus and method.

FIG. 3 is a side view of the hygiene product illustrated in FIG. 2.

FIGS. 4A and 4B are perspective and top schematic views of a processing system for preparing and re-orienting discrete hygiene products for packaging, according to an embodiment of the invention.

FIG. 5 is a flow chart illustrating a method of re-positioning folded hygiene products for packaging, according to embodiments of the invention.

FIG. 6A is a side view showing folded discrete hygiene products in vertical orientation and re-oriented and rotated +/−90°, with alternating discrete hygiene products rotated +90° and −90°, according to an embodiment of the invention.

FIG. 6B is a top schematic view of the system of FIGS. 3A and 3B operating to place the discrete hygiene products in the arrangement illustrated in FIG. 6A.

FIG. 7A is a side view showing folded discrete hygiene products in vertical orientation, with alternating discrete hygiene products left unrotated at 0° and rotated 180°.

FIG. 7B is a top schematic view of the system of FIGS. 3A and 3B operating to place the discrete hygiene products in the arrangement illustrated in FIG. 7A.

FIG. 8A is a side view showing folded discrete hygiene products in vertical orientation and re-oriented and rotated, with each discrete hygiene product rotated −90°, according to an embodiment of the invention.

FIG. 8B is a top schematic view of the system of FIGS. 3A and 3B operating to place the discrete hygiene products in the arrangement illustrated in FIG. 8A.

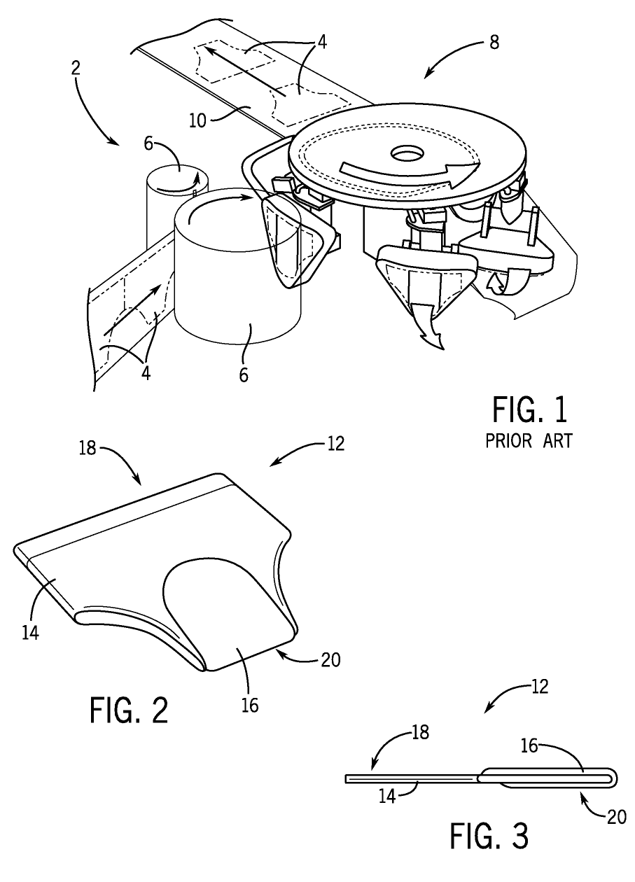

### DETAILED DESCRIPTION

Embodiments of the present invention provide for a method and apparatus for mechanically manipulating traveling discrete hygiene products to re-position them in a desired orientation for packaging. The orientation of each of a plurality of successive discrete hygiene products may be selectively controlled in order to re-position the discrete hygiene products in any of a number of desired orientations.

Referring first to FIGS. 2 and 3, perspective and side views of a diaper 12 are provided, with the diaper being exemplary of a discrete hygiene product that may be processed and handled for packaging according to systems and methods of the present invention. While a diaper 12 is illustrated in FIGS. 2 and 3 and described hereinafter in relation to embodiments of the invention, it is recognized that embodiments of the invention extend to re-orienting and packaging other discrete hygiene products, especially those with varying thicknesses, and are not limited only to use with such diapers.

As shown in FIGS. 2 and 3, diaper 12 is generally composed of a chassis 14 and absorbent insert or core 16. While not shown, it is understood that the chassis 14 may comprise layers of the nonwoven web material that sandwich a support structure that is generally elastic and may include leg elastic, waistband elastic, and belly band elastic. The chassis 14 may also be provided with several patches, besides the absorbent core 16, such as adhesive tape tabs and resealable closures. The absorbent core 16 may be composed of cellulose pulp core that includes a super-absorbent powder that functions to absorb exudates to keep the wearer dry.

As best illustrated in FIG. 3, a waist region 18 of the diaper 12 is generally thinner that a crotch region 20 of the diaper 12 due to the presence of the absorbent core 16. This mismatch between a thickness of the waist region 18 and the crotch region 20 is magnified upon folding of the diaper 12 into a final form prior to packaging. Accordingly, it is recognized that—in packaging a plurality of diapers 12 in a stacked arrangement—orienting each of the plurality of diapers 12 in a same/common manner may result in a less efficient final packaging. That is, as the thicker crotch regions 20 of the diapers 12 are all stacked on top of one another, the height of the stack may be higher than necessary as compared to if the crotch region 20 of adjacent diapers 12 was alternated in the stack such that the thicker crotch region 20 and thinner waist region 18 of the diapers 12 nest together, providing a more compact and efficient packaging.

According to embodiments of the invention, an apparatus and method are provided that re-orient discrete hygiene products travelling in a machine direction. The apparatus and method re-orients the discrete hygiene products by turning and placing the discrete hygiene products in a pre-determined arrangement. Such an arrangement may include rotating each discrete product −90°, rotating every other discrete product 180°, and/or rotating consecutive discrete products +90° and −90° in opposite directions, for example. The re-orienting of the products may be selectively controlled and modified to achieve a desired final packaging arrangement, so as to achieve maximum efficiency in packaging.

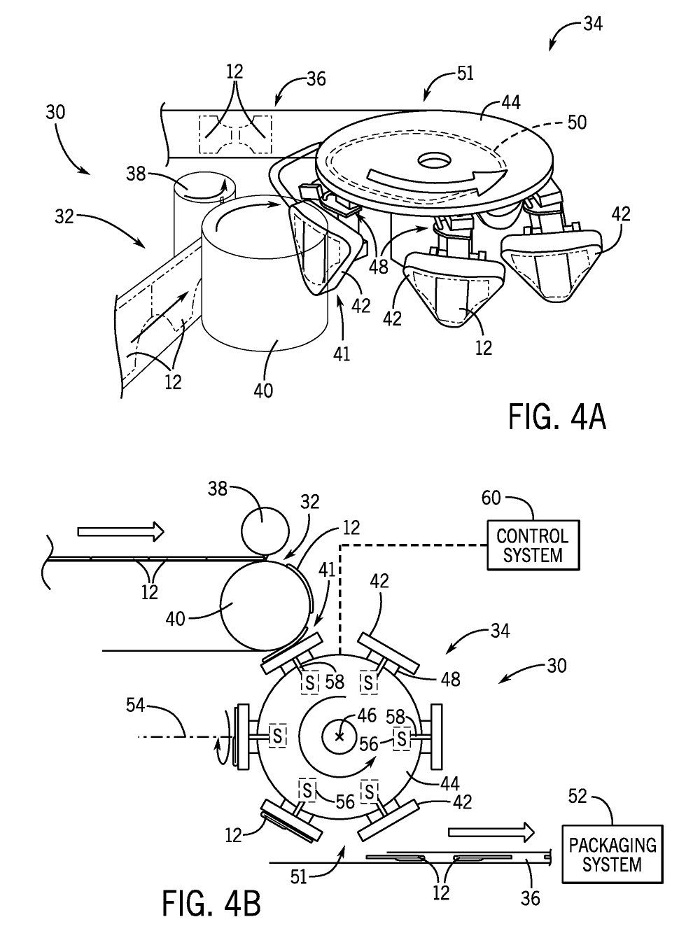

Referring now to FIGS. 4A and 4B, a processing system 30 for preparing and re-orienting discrete hygiene products—e.g., diapers 12—in preparation for packaging is shown, according to an embodiment of the invention. The system 30 generally comprises an input device 32, a product turner and placer 34 positioned to receive the discrete diapers from the input device 32, and a conveyor 36 positioned adjacent the product turner and placer 34 to receive discrete diapers 12 therefrom.

In FIGS. 4A and 4B, an incoming web such as a web of continuous formed and sideseam bonded diapers 12 is shown entering a vertically oriented knife 38 and anvil 40 arrangement (of input device 32). The sideseam bonded diapers 12 may be provided to input device 32 as diapers that are already in a folded state, and the knife 38 and anvil 40 arrangement cooperate to sever the bonded diapers to separate them into discrete diapers, with the diapers 12 being translated about a surface of anvil 40 before being transferred to product turner and placer 34.

The product turner and placer 34 acquires the diapers 12 from the anvil 40 at an acquisition point 41. The product turner and placer 34 includes a series of pucks 42 that are rotatably mounted, carried, and rotated by a rotating unit or puck wheel 44 that rotates about a central axis 46. According to an embodiment of the invention, the pucks 42 are carried by arms 48 that are coupled to puck wheel 44. The radial and lateral position of the pucks 42 can be selectively controlled via cam followers (not shown, but on arms 48) and an interaction thereof with cam slots (e.g. cam slot 50). As the pucks 42 rotate, the cam followers travel through the eccentric cam slot 50 and adjust the lateral position of the pucks 42, as desired, so as to control/change the relative rotational speed of the pucks 42.

The pucks 42 are preferably vacuum pucks to retain the diapers 12 upon a carrying surface of the pucks 42 and selectively release them at a desired time. However, other attraction or retaining forces can be employed to retain the diapers 12 on the carrying surface of the pucks 42. After acquiring a diaper 12 at acquisition point 41 and rotating about puck wheel 44 to a deposition point 51, each of the pucks 42 releases an individual diaper 20 onto conveyor 36, which then transfers the diapers 12 downstream to a packaging apparatus 52 that subsequently packages the diapers 12 in a desired fashion. Continuing their revolution, pucks 42 then continue to rotate past the deposition point 51 and back to the acquisition point 41 to acquire another diaper 12 from input device 32 to their original vertical orientation upon returning to the acquisition point 41 proximal to anvil 40.

In addition to rotating pucks 42 about a central axis 46 to transfer discrete diapers 12 from acquisition point 41 to deposition point 51, each of the pucks 42 is further operable to rotate or spin about a spin axis 54 thereof, so as to re-orient the diapers 12 in a desired manner for placement on the conveyor 36 and subsequent packaging. That is, each of pucks 42 is independently controllable to spin about its own spin axis 54 in a desired direction and by a desired amount (i.e., by a desired angle). For effectuating this spinning of the pucks 42, any of a number of suitable mechanical constructions of the product turner and placer 34 may be implemented, and it is recognized that specific structures and mechanisms described here below are not meant to limit the scope of the invention.

According to one embodiment, the rotation of each of pucks 42 about its respective spin axis 54 can be achieved via associated servo drives 56, each of which may control the relative rotational movement of a puck 42 about its respective spin axis 54. The servo drives 56 may be secured to the puck wheel 44 and operable to drive a rotatable shaft 58 that may be coupled to a linkage or gearing (not shown) that causes rotation of the puck about the spin axis 54. The servo drives 56 are preferably controlled by and communicatively coupled to a servo drive control system 60 that sends individual control signals thereto, so as to provide for independent and differential operation of each of the servo drives 56.

According to an alternative embodiment, rotational modification of the pucks 42 may be achieved via interaction between a spin cam follower and spin race (not shown) formed on a cam plate (not shown). A spin linkage (not shown) couples the puck 42 to the spin cam follower, with the spin cam follower sliding or rolling within the spin cam race (e.g., such as via a roller bearing) to provide for rotation of the puck 42 about the spin axis 54 in a desired direction and by a desired amount.

As shown in FIGS. 4A and 4B, the processing system 30 is configured to receive the incoming web of continuous formed and sideseam bonded diapers 12 as a vertically oriented web at input device 32 and to output discrete diapers 12 in a vertical orientation onto a vertical conveyor 36. This vertical input to vertical output configuration beneficially allows for a reduction in the overall footprint of the processing system 30. That is, as previously described, outputting of the diapers 12 in a vertical orientation to a vertical conveyor 36 for packaging—rather than outputting the diapers 12 in a horizontal orientation to a horizontal conveyor and then having to re-orient them to vertical for packaging, as is conventionally done—allows for a process step to be eliminated and for the conveyor length to be shortened. It is recognized, however, that embodiments of the invention could output diapers 12 in a horizontal orientation to a horizontal conveyor rather than outputting diapers in a vertical orientation. Such an embodiment would not result in a more compact footprint for the system 30, but would still re-orient the diapers 12 by turning and placing the diapers 12 in a pre-determined arrangement on the horizontal conveyor, so as to achieve a desired final packaging arrangement with maximum efficiency in packaging.

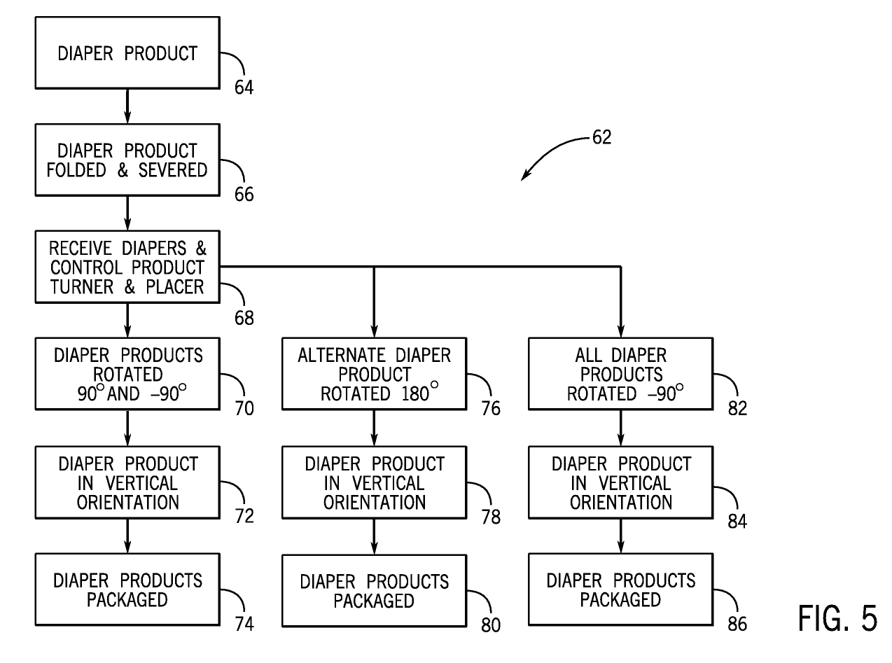

Referring now to FIG. 5, a flowchart illustrating a method 62 for manipulating traveling discrete hygiene products (e.g., diapers) to re-position them in a desired orientation for packaging is provided, according to an embodiment of the invention. The method 62 may be carried out by a system such as the system illustrated in FIGS. 4A and 4B, components of which are referred to hereafter in conjunction with description of the method 62.

In beginning the method 62, a plurality of diaper products is provided at STEP 64. The diapers 12 may be fabricated and assembled according to known methods, with an absorbent insert and a chassis being fabricated and assembled from a plurality of materials/components, include nonwoven web material(s), elastic materials, cellulose pulp, super-absorbent material, and patches (adhesive tape tabs and resealable closures). The diapers 12 may be fabricated such that they are still joined as a web of continuously formed and sideseam bonded diapers.

Upon fabrication/assembly of the diapers 12, the diapers are subsequently folded at the crotch region thereof as indicated at STEP 66, such that the web of continuously formed and sideseam bonded diapers 12 are in a folded state. The folding of diapers 12 may be performed via a known method, with the folded diaper thus having a crotch region 20 that is thicker than a waist region 18 thereof due to the presence of the absorbent core or insert 16 that is located in the crotch region. Also at STEP 66, and subsequent to folding of the diapers 12, the web of continuously formed and sideseam bonded diapers 12 is severed so as to provide discrete diapers that are ready for packaging. According to one embodiment, the web of continuously formed and sideseam bonded diapers 12 is severed at an input device 32 via a knife 38 and anvil 40 arrangement thereof. According to one embodiment, the web of continuously formed and sideseam bonded diapers 12 (in a folded state) is provided to input device 32 for severing in a vertical orientation, as illustrated in FIGS. 4A and 4B.

Upon completion of the folding and severing operations performed at STEP 66, the discrete diapers 12 are received at the product turner and placer 34 at STEP 68. According to an exemplary embodiment, each discrete diaper 12 is received on a puck 42 of the product turner and placer 34—with each puck 42 securely holding a diaper 12 thereon, such as via a vacuum system and/or other attraction or retaining forces. Also at STEP 68, the product turner and placer 34 is operated in order to achieve a selected manipulation of the diapers 12 for placement on conveyor 36 and subsequent packaging of the diapers. Operation of the product turner and placer 34 may be determined via a pre-programming of a control system 60 associated with the product turner and placer 34, with it being recognized that the control system 60 could be programmed to operate the product turner and placer 34 in any one of a number of distinct operating modes. Based on the selected operating mode, the control system 60 thus functions to operate the product turner and placer 34 to control the relative rotational speed of the pucks 42 about central axis 46 (to control a pitch of pucks 42), as well as control rotation of each of the pucks 42 about their respective spin axes 54. The control system 60 may thus control the product turner and placer 34 to re-orient discrete diapers 12 travelling in a machine direction and in a vertical position by turning and placing the discrete diapers in a pre-determined arrangement (and at a pre-determined pitch) on conveyor 36 for packaging. Such an arrangement may include rotating each discrete diaper −90°, rotating every other discrete diaper 180°, or rotating a pair of successive diapers +90° and −90° in opposite directions. The distinct methods of operation of the product turner and placer 34 indicated above are now described here in greater detail, according to embodiments of the invention.

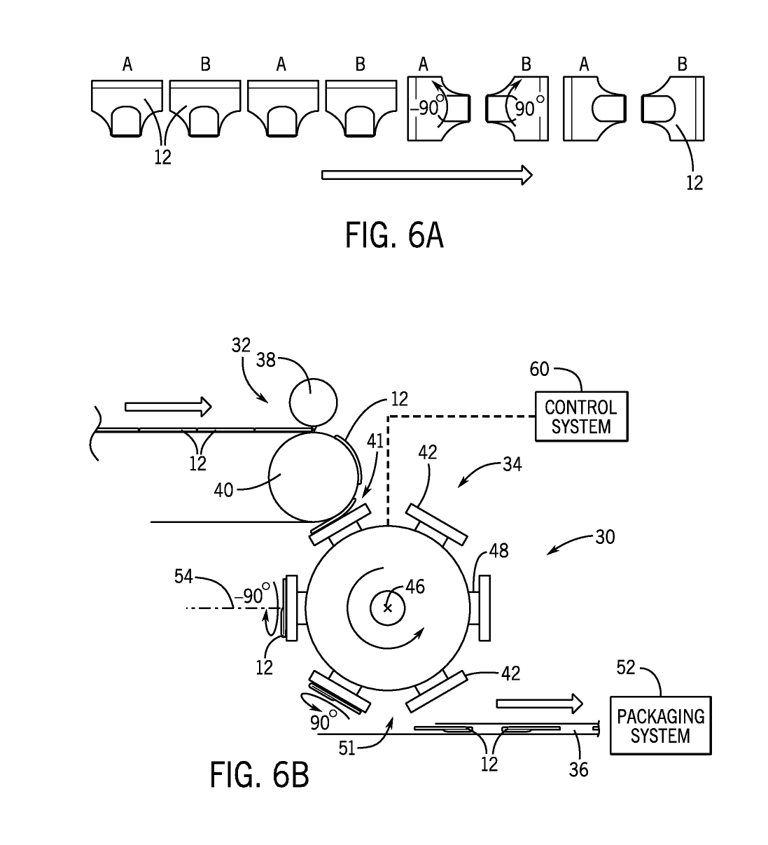

Referring to STEPS 70-74 in FIG. 5 and now also to FIGS. 6A and 6B, operation of the product turner and placer 34 according to a first operating mode is described. As shown in FIGS. 6A and 6B, diapers 12 are provided in a first orientation, with an incoming web of continuous formed and sideseam bonded diapers 12 (that have previously been folded at the crotch region) being severed by the knife 38 and anvil 40 arrangement such that discrete diapers 12 are subsequently provided to product turner and placer 34. Control system 60 then causes product turner and placer 34 to operate according to a first operating mode, including controlling rotation of each of the pucks 42 about their respective spin axes 54. In the first operating mode, each successive pair of diapers 12 are rotated by +90° and −90° in an opposite direction. That is, a first puck 42 is rotated +90° and a second puck 42 adjacent the first puck 42 is rotated −90°, such that a pair of immediately adjacent discrete diapers 12 are deposited on the conveyor 36 in opposite orientations, with a crotch region 20 and waist region 18 of the diapers aligned along the machine direction (STEP 70). As the pucks 42 rotate to re-orientate the discrete diapers 12 in alternating orientations, the pucks 42 also maintain the diapers 12 in a vertical orientation (STEP 72). The diapers 12 are then passed to a packaging system 52 for packaging thereof (STEP 74), with the alternating orientations of the diapers 12 allowing the diapers to nest together and thereby provide a more compact and efficient packaging.

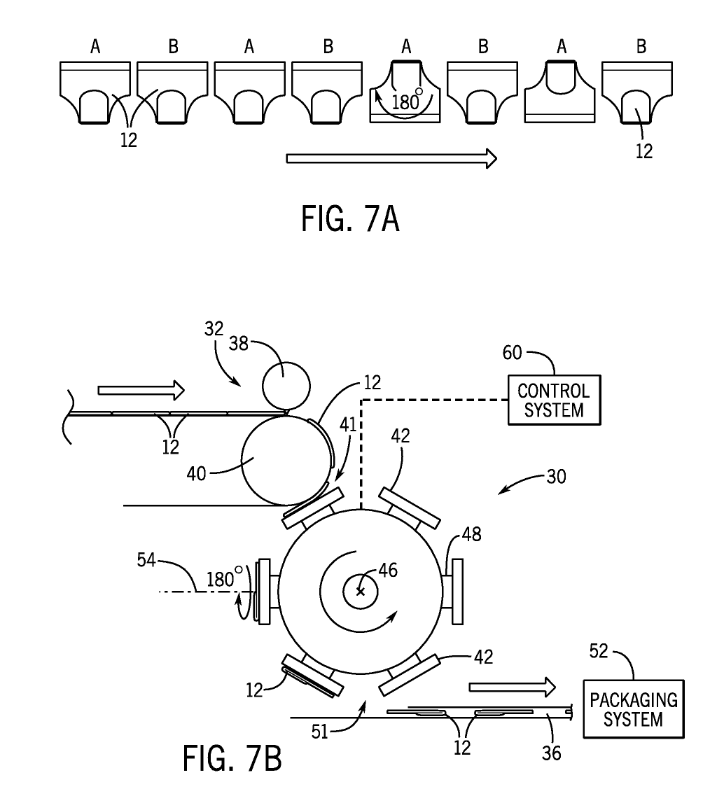

Referring to STEPS 76-80 in FIG. 5 and now also to FIGS. 7A and 7B, operation of the product turner and placer 34 according to a second operating mode is described. As shown in FIGS. 7A and 7B, diapers 12 are provided in a first orientation, with an incoming web of continuous formed and sideseam bonded diapers 12 (that have previously been folded at the crotch region) being severed by the knife 38 and anvil 40 arrangement such that discrete diapers are subsequently provided to product turner and placer 34. Control system 60 then causes product turner and placer 34 to operate according to a second operating mode, including controlling rotation of each of the pucks 42 about their respective spin axes 54. In the second operating mode, every other puck 42 is rotated by 180°, with intervening pucks 42 left unrotated (i.e., “rotated” by or left at 0°), such that a pair of immediately adjacent diapers 12 are deposited on the conveyor 36 in opposite orientations, with a crotch region 20 and waist region 18 of the diapers 12 orthogonal to the machine direction (STEP 76). As the pucks 42 are controlled to re-orientate the discrete diapers 12 in alternating orientations, the pucks 42 also maintain the diapers 12 in a vertical orientation (STEP 78). The diapers 12 are then passed to a packaging system 52 for packaging thereof (STEP 80), with the alternating orientations of the diapers 12 allowing the diapers to nest together and thereby provide a more compact and efficient packaging.

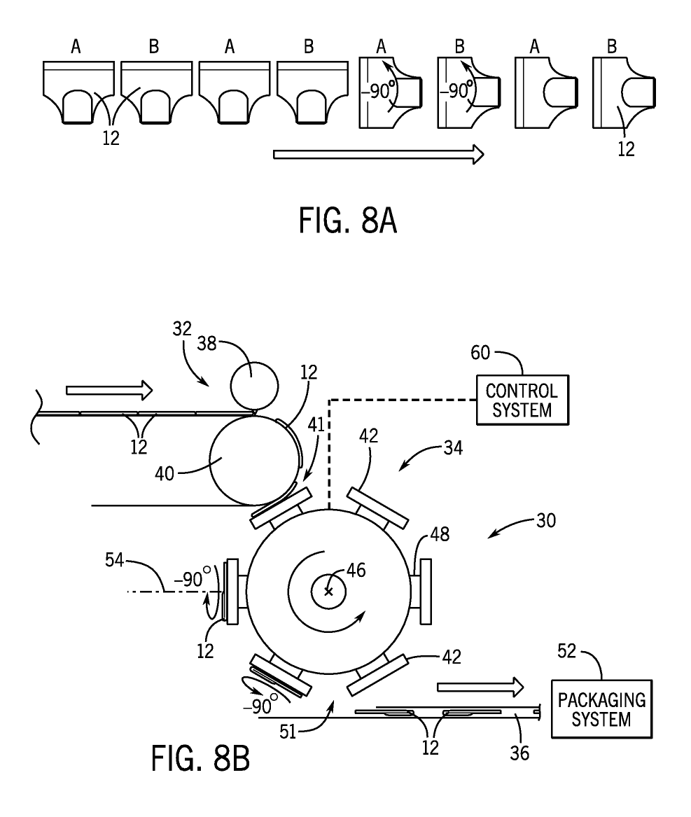

Referring to STEPS 82-86 in FIG. 5 and now also to FIGS. 8A and 8B, operation of the product turner and placer 34 according to a third operating mode is described. As shown in FIGS. 8A and 8B, diapers 12 are provided in a first orientation, with an incoming web of continuous formed and sideseam bonded diapers 12 (that have previously been folded at the crotch region) being severed by the knife 38 and anvil 40 arrangement such that discrete diapers 12 are subsequently provided to product turner and placer 34. Control system 60 then causes product turner and placer 34 to operate according to a third operating mode, including controlling rotation of each of the pucks 42 about their respective spin axes 54. In the third operating mode, each puck 42 is rotated in the same direction and by a same angular amount. According to an exemplary embodiment, each puck 42 is rotated by −90° (STEP 82), such that the discrete diapers 12 are deposited on the conveyor 36 with a crotch region 20 of each diaper as the leading edge of the product and aligned along the machine direction. It is recognized, however, that each puck 42 could instead be rotated by +90°, such that the discrete diapers 12 are deposited on the conveyor 36 with a crotch region 20 of each discrete diaper as the trailing edge of the product and aligned along the machine direction. As the pucks 42 are controlled to re-orientate the discrete diapers 12, the pucks 42 also maintain the diapers 12 in a vertical orientation (STEP 84). The diapers 12 are then passed to a packaging system 52 for packaging thereof (STEP 86).

Thus, according to method 62—and the various distinct operating modes that may be performed therein—discrete diapers 12 are re-oriented from a first vertical orientation entering knife 38 and anvil 40 combination to other vertical orientations when output to conveyor 36. The discrete diapers 12 are re-oriented from a uniform vertical orientation to one or more re-positioned vertical orientations by rotation/spinning of pucks 42, with the method allowing for selection and customization of controlling rotation of the diapers for packaging.

While embodiments of the invention are described above as being performed by a single product turner and placer 34, it is recognized that multiple product turner and placers (e.g., two) could be utilized in order to re-orient discrete diapers from a first orientation to other orientation(s), so as to provide for efficient packaging thereof. In such an embodiment, a first product turner and placer 34 would operate to perform a first axis movement while a second product turner and placer 34 (positioned downstream from the first product turner and placer 34) would operate to perform a second axis movement. The first and second product turner and placers could thus collectively perform a two-axis movement, according to an embodiment of the invention.

Beneficially, embodiments of the invention thus provide a system and method for mechanically manipulating traveling discrete hygiene products to re-position them in a desired orientation for packaging. The orientation of each of a plurality of successive discrete hygiene products may be selectively controlled in order to re-position the discrete hygiene products in any of a number of desired orientations, including orientations that allow for products to be nested together to provide a more compact and efficient packaging. Additionally, the system and method of the present invention comprise a vertical input to vertical output product process flow that enables a length of the packaging line to be shortened based on the elimination of an additional turning/rotating step, such that the packaging system footprint may be reduced.

Therefore, according to one embodiment of the invention, a method for reorienting discrete hygiene products for packaging is provided. The method includes providing discrete hygiene products traveling in a machine direction and all in a first orientation to a product turner and placer, the product turner and placer comprising a plurality of pucks attached to a puck wheel so as to be rotatable about a central axis thereof, with each puck also rotatable about a spin axis thereof. The method also includes receiving a discrete hygiene product on each respective puck, transferring the discrete hygiene products via the plurality of pucks from a pick-up location to a drop-off location, and depositing the discrete hygiene products from the plurality of pucks onto a conveyor at the drop-off location, the discrete hygiene products deposited so as to be spaced apart on the conveyor. In transferring and depositing the discrete hygiene products, the method further comprises selectively controlling the rotation of each of the plurality of pucks about its spin axis to reorient each of the discrete products for depositing onto the conveyor, with each of a direction and an amount of the rotation being selectively controlled.

According to another embodiment of the invention, a discrete hygiene product processing system includes an input device configured to translate discrete hygiene products in a machine direction and a product turner and placer positioned to receive the discrete hygiene products from the input device. The product turner and placer further includes a puck wheel rotatable about a central axis and a plurality of pucks attached to the puck wheel so as to be rotatable therewith, with each of the plurality of pucks configured to discretely rotate about a spin axis and configured to receive a discrete hygiene product from the input device. The discrete hygiene product processing system also includes a conveyor positioned adjacent the product turner and placer to receive discrete hygiene products as they are released from the plurality of pucks. Each of the plurality of pucks is selectively controllable to rotate about its spin axis to reorient each of the discrete products for depositing onto the conveyor.

While the invention has been described in detail in connection with only a limited number of embodiments, it should be readily understood that the invention is not limited to such disclosed embodiments. Rather, the invention can be modified to incorporate any number of variations, alterations, substitutions or equivalent arrangements not heretofore described, but which are commensurate with the spirit and scope of the invention. Additionally, while various embodiments of the invention have been described, it is to be understood that aspects of the invention may include only some of the described embodiments. Accordingly, the invention is not to be seen as limited by the foregoing description, but is only limited by the scope of the appended claims.

What is claimed is:

1. A method for reorienting discrete hygiene products for packaging, the method comprising:
providing discrete hygiene products traveling in a machine direction and all in a first orientation to a product turner and placer, the product turner and placer comprising a plurality of pucks attached to a puck wheel so as to be rotatable about a central axis thereof, with each puck also rotatable about a spin axis thereof;
receiving a discrete hygiene product on each respective puck;
transferring the discrete hygiene products via the plurality of pucks from a pick-up location to a drop-off location; and
depositing the discrete hygiene products from the plurality of pucks onto a conveyor at the drop-off location, the discrete hygiene products deposited so as to be spaced apart on the conveyor;
wherein, in transferring and depositing the discrete hygiene products, the method comprises selectively controlling the rotation of each of the plurality of pucks about its spin axis to reorient each of the discrete products for depositing onto the conveyor, with each of a direction and an amount of the rotation being selectively controlled.

2. The method of claim 1 wherein selectively controlling the rotation of each of the plurality of pucks comprises rotating a first puck +90° and rotating a second puck adjacent the first puck −90°, such that a pair of immediately adjacent discrete hygiene products are deposited on the conveyor in opposite orientations, with a crotch region and waist region of the discrete hygiene products aligned along the machine direction.

3. The method of claim 1 wherein selectively controlling the rotation of each of the plurality of pucks comprises rotating every other puck of the plurality of pucks by 180°, such that a pair of immediately adjacent discrete hygiene products are deposited on the conveyor in opposite orientations, with a crotch region and waist region of the discrete hygiene products orthogonal to the machine direction.

4. The method of claim 1 wherein selectively controlling the rotation of each of the plurality of pucks comprises rotating each of the plurality of pucks −90°, such that the discrete hygiene products are deposited on the conveyor such that a crotch region of each discrete hygiene product is a leading edge of the product and aligned along the machine direction.

5. The method of claim 1 wherein selectively controlling the rotation of each of the plurality of pucks comprises receiving an operator input selecting a desired rotation of each of the plurality of pucks.

6. The method of claim 1 further comprising:
translating the discrete hygiene products along the conveyor in the machine direction to a packaging station; and
stacking the discrete hygiene products at the packaging station for securing in a packaging material.

7. The method of claim 1 further comprising folding the discrete hygiene products prior to providing the discrete hygiene products to the product turner and placer.

8. The method of claim 1 wherein receiving a discrete hygiene product on each respective puck comprises securing a discrete hygiene product on each respective puck via a vacuum.

9. The method of claim 1 further comprising:
providing an incoming continuous web of material traveling in the machine direction along a vertical plane of travel; and
severing the web into the discrete hygiene products.

10. The method of claim 9 wherein the incoming continuous web of material is provided in a vertical orientation, and wherein the discrete hygiene products are deposited onto the conveyor in a vertical orientation.

11. The method of claim 1 wherein the product turner and placer comprises a first product turner and placer, and wherein the method further comprises providing the discrete hygiene products to a second product turner and placer downstream from the first product turner and placer; and
wherein, in transferring and depositing the discrete hygiene products, the first product turner and placer performs a first rotation or axis movement and the second product turner and placer performs a second rotation or axis movement.

12. A discrete hygiene product processing system comprising:
an input device configured to translate discrete hygiene products in a machine direction;
a product turner and placer positioned to receive the discrete hygiene products from the input device, the product turner and placer comprising:
a puck wheel rotatable about a central axis; and
a plurality of pucks attached to the puck wheel so as to be rotatable therewith, with each of the plurality of pucks configured to discretely rotate about a spin axis;
wherein each of the plurality of pucks is configured to receive a discrete hygiene product from the input device; and
a conveyor positioned adjacent the product turner and placer to receive discrete hygiene products as they are released from the plurality of pucks;
wherein each of the plurality of pucks is selectively controllable to rotate about its spin axis to reorient each of the discrete products for depositing onto the conveyor.

13. The system of claim 12 further comprising a plurality of servo drives, each of the plurality of servo drives controlling the rotation of a respective puck about its spin axis by turning a rotatable shaft to which the puck is attached.

14. The system of claim 13 wherein a first servo drive rotates a first puck +90° and a second servo drive rotates a second puck adjacent the first puck −90°, such that a pair of immediately adjacent discrete hygiene products are deposited on the conveyor in opposite orientations, with a crotch region and waist region of the discrete hygiene products aligned along the machine direction.

15. The system of claim 13 wherein a first servo drive remains inactive on a first puck to leave the puck at 0° and a second servo drive rotates a second puck adjacent the first puck −180°, such that a pair of immediately adjacent discrete hygiene products are deposited on the conveyor in opposite orientations, with a crotch region and waist region of the discrete hygiene products orthogonal to the machine direction.

16. The system of claim 13 wherein each of the plurality of servo drives rotates a respective puck −90°, such that the discrete hygiene products are deposited on the conveyor such that a crotch region of each discrete hygiene product is a leading edge of the product and aligned along the machine direction.

17. The system of claim 13 further comprising a control system operatively coupled to the plurality of servo drives to control operation thereof, so as to control a direction and amount of rotation of each of the plurality of pucks.

18. The system of claim 12 wherein the control system is programmed to receive an operator input selecting a desired rotation of each of the plurality of pucks.

19. The system of claim 12 wherein the input device provides the discrete hygiene products to the product turner and placer in a vertical orientation, and wherein the product turner and placer deposits the discrete hygiene products onto the conveyor in a vertical orientation.

20. The system of claim 12 wherein the input device is configured to sever an incoming continuous web of material into the discrete hygiene products and fold the discrete hygiene products prior to the product turner and placer receiving the discrete hygiene products.
